(19) United States
(12) Patent Application Publication
(10) Pub. No.: US 2026/0153507 A1
(43) Pub. Date: Jun. 4, 2026

(54) DIFFUSIONAL MICROFLUIDICS

(71) Applicant: NUCLERA LTD, Cambridge (GB)

(72) Inventors: Sumit KALSI, Cambridge (GB); Gordon Ross MCINROY, Cambridge (GB); Richard J. PAOLINI, Jr., Cambridge (GB); Luke M. SLOMINSKI, Cambridge (GB); Andrea ZANZOTTO, Cambridge (GB)

(73) Assignee: NUCLERA LTD, Cambridge (GB)

(21) Appl. No.: 18/707,435

(22) PCT Filed: Nov. 7, 2022
(86) PCT No.: PCT/GB2022/052801

§ 371 Date: May 3, 2024

(30) Foreign Application Priority Data
Nov. 5, 2021 (GB) 2115946.2

(51) Int. Cl. G01N 33/558; G01N 33/58

(52) U.S. Cl. CPC G01N 33/558; G01N 33/582

(57) ABSTRACT

Provided herein is a method for measuring the properties of biomolecules by measuring the diffusion within a merged droplet on a microfluidic device.

### FIELD OF THE INVENTION

Provided herein is a method for measuring the diffusion within a merged droplet on a microfluidic device.

### BACKGROUND TO THE INVENTION

There is an ongoing interest in assays that measure the stability, affinity or structural changes in proteins. For example, by performing a thermal shift assay (TSA) whereby the protein of interest is heated in the presence of fluorescent agent SYPRO Orange and fluorescence is monitored as the protein unfolds and the dye binds. One issue with the TSA is that it measures the thermal stability of a protein, which may not correlate directly with the room temperature/moderate temperature stability that is more relevant for biological interactions. Another issue is that TSA typically requires heating up to 70-90 degrees Celsius. This is not well-suited to technology implemented on conventional microfluidic devices which are not thermally stable. Working on small reagent volumes at high temperatures is problematic due to evaporation or gas evolution.

Diffusional sizing methods are known in the context of channel flow microfluidic devices made from PDMS using soft lithography. An example publication is Arosio et. al. ACS Nano 2016, 10, 1, 333-341 (Microfluidic Diffusion Analysis of the Sizes and Interactions of Proteins under Native Solution Conditions https://doi.org/10.1021/acsnano.5b04713), which describes implementation of a method for measuring the hydrodynamic radius by measuring diffusion of a flow focused analyte stream in a soft-lithography flow-based microfluidic device. Measurement of diffusion at multiple time points simultaneously is made by measuring at multiple points along the flow channel. Fluorescent diffusion profiles generated are symmetric gaussian profiles due to a central analyte and “blank” solutions either side. Such methods can only be implemented one at a time, one profile per experiment, there is no ability to multiplex assays or reagents. A high throughput system for measuring biomolecule stability and interactions using diffusion rates is therefore to be desired.

EP3695905 describes a system for separating differently sized molecules based on the rate of diffusion.

EP3812041 refers to microfluidic channel devices and their use in a variety of applications.

### SUMMARY OF THE INVENTION

Provided herein is a method for measuring the diffusion of fluorescent reagents within a merged droplet on a microfluidic device. More specifically, a method for measuring the diffusion within a merged droplet on an electrowetting on dielectric (EWoD) device. After merging, the droplets engage in non-turbulent mixing. The rate of mixing allows measurement of the properties of the biomolecules such as affinity to external ligands, denaturing or aggregation. For example the rate of diffusion can be measured in droplets having different ligands or varying ion concentrations or pH. For example fluorescent affinity antibodies can be used to see if certain proteins are present causing the fluorescent antibodies to aggregate or diffuse more slowly.

Measurement of the hydrodynamic radius informs on protein stability as many pathways to instability can be assessed. E.g. degradation will cause a decrease in apparent hydrodynamic radius, while aggregation will cause an increase. The hydrodynamic radius can be calculated from mostly known parameters and the diffusion coefficient which can be determined by observing the diffusion profile of an analyte, e.g. via tracking fluorescence. Using this approach in digital microfluidic (DMF) devices enables many measurements in parallel and enables microfluidic diffusional sizing (MDS) to be used in a much higher throughput manner than is currently available.

Disclosed herein is a method for measuring the diffusion within a merged droplet on an electrowetting on dielectric (EWoD) device, the method comprising:
a. adding at least two droplets onto the device, one droplet containing a detectable analyte and the second droplet containing an aqueous solution;
b. manipulating two droplets to merge the detectable analyte and aqueous solution; and
c. measuring the diffusion of the detectable analyte within the merged droplet and calculating the diffusion coefficient of the detectable analyte.

The detectable analyte may be a biomolecule. The biomolecule may be a protein or antibody. The biomolecule or protein may be fluorescent. For example, the fluorescent protein may be green fluorescent protein (GFP) or a protein labelled with fluorescent labels. The method may be performed on many droplets in parallel or in a time series.

### BRIEF DESCRIPTION OF THE FIGURES

FIG. 1 shows (A) two droplets present on a digital microfluidic (DMF) device. The black droplet represents a fluorescent droplet containing fluorescein, GFP or another fluorescent biomolecule, e.g. a fluorescently labelled protein. The white droplet represents a blank, non-fluorescent, droplet containing only a buffer. (B) the two droplets are brought together to form a single droplet. (C) in DMF devices diffusion is the primary form of mixing; the fluorescent biomolecule will diffuse into the blank (white) droplet. (D)-(F) the diffusion (rightmost part of fluorescent moiety) progressively moves into the blank (white) droplet. The rate of mixing is dependent on the hydrodynamic radius (i.e. size in solution) of the molecules.

FIG. 2 shows cartoon example fluorescence profiles derived from a left hand fluorescent drop and a right hand blank droplet meeting. The solid line shows the profile just after mixing, where fluorescence is only detected in the left droplet. The dashed line shoes the profile after some diffusion has occurred, and the dotted line shows the profile after more diffusion has occurred.

FIG. 3 shows the merging of two droplets with different geometries. Being able to control the geometries of each droplet may aid in minimising turbulent mixing upon droplet merging.

FIG. 4 shows a protein diffusion script. (a) Droplets are dispensed, (b) droplets are re-shaped into rectangles, (c) droplets are brought together in pairs, with each pair consisting of a droplet containing fluorescent molecules and a droplet containing buffer.

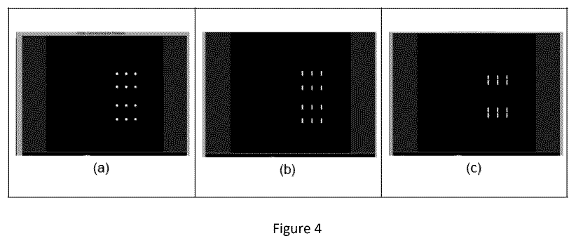

FIG. 5 shows time lapse images of molecular diffusion between adjacent droplets of fluorescent molecules and buffer (20 mM HEPES-KOH pH 7.2, 50 mM NaCl, 0.1% v/v Tween 20). It can be seen that diffusion of deGFP and Atto 488-Biotin begins immediately after droplet contact and continues at every time point thereafter.

FIG. 6 shows enlarged images of droplets after 14 minutes of continuous actuation. In the enlarged images, a difference can be seen in the diffusion profile of deGFP (left) and Atto 488-Biotin (right). The larger deGFP (left) can be seen to have diffused less than the smaller Atta 488-biotin (right).

FIG. 7 shows how droplet relaxation and actuation can be employed to accelerate mixing of the fluorescent species in a size-dependent manner. A time series of images taken (a) before (b) during and (c) after the first droplet relaxation, as well as (d) during and (e) after the second droplet relaxation. After the initial period of continuous actuation, two “relaxations” were carried out during which actuation of all droplets was paused and they were allowed to sit in place for three minutes. After each pause in actuation, actuation was resumed using the identical shape and size of each droplet as before. FIG. 7 illustrates the state of the droplets during and after each actuation pause. The difference in behaviour between the deGFP and Atto 488 is even more clearly apparent in the above series of droplet actuation/relaxation cycles. In image (e) above, it can be seen that the Atto 488 dye now fills the entire combined droplet region, while the deGFP is still found only on the side on which it was originally dispensed. In addition to differences in diffusion rates, convective mass transfer in the form of mixing may have contributed to this difference in spatial distribution. Fluorescent imaging of mixing patterns and rates may therefore also be a useful tool in determining the molecular weight of a given reagent.

FIG. 8 shows quantitatively the difference in diffusion profiles of a small molecule fluorophore (Atto 488) and a larger protein (GFP). The larger species diffuse into the blank droplet at a slower rate than the smaller species.

### DETAILED DESCRIPTION OF THE INVENTION

Provided herein is a method for measuring the diffusion of species within a merged droplet on an electrowetting on dielectric (EWoD) device, the method comprising:
a. adding at least two droplets onto the device, one droplet containing a detectable analyte and the second droplet containing an aqueous solution;
b. manipulating two droplets to merge the analyte and aqueous solution; and
c. measuring the diffusion of the detectable analyte within the merged droplet and calculating the diffusion coefficient of the detectable analyte.

The detectable analyte may be a single analyte species in each droplet having a detectable analyte. The phrase single analyte refers to for example a labelled protein which may be in a variety of conformational or aggregated states. The phrase also refers to for example a labelled protein or nucleic acid population which may be partially bound to a ligand or to itself or to a hybridization partner. The method is aimed at measuring diffusion coefficients, in order to measure for example the folding or aggregation of proteins in varying conditions or over time or the level of binding of a fluorescent protein to a ligand or whether a nucleic acid is single or double stranded.

The detectable analyte may be a micro or nanoparticle. The analyte may be a species such as a virus. The analyte may be a biomolecule. The biomolecule may be a protein. The biomolecule may be a nucleic acid. The analyte may be inherently fluorescent, such as GFP, or be fluorescently labelled. The fluorescent analyte may be a biomolecule bound to a fluorescent marker.

The analyte may be an antibody, thus making an assay for detecting the presence of unlabeled binding agents, which cause the antibody to diffuse at a different rate. For example the analyte may be an antibody for detecting a virus or other biological moiety of interest. Thus included is a method wherein the presence of non-fluorescent species are identified by the change in diffusion of the fluorescent species. In other words, the detectable analyte in one droplet can be used to determine the presence or absence of a non-detectable analyte in the blank droplet. In such instances comparison with a control aqueous droplet where the non-detectable analyte is not present may be used.

The detectable analyte may be a small particle in solution, for example, a colloid in solution.

The analyte may be detected by measuring absorbance or through fluorescence detection if the analyte is inherently fluorescent or is fluorescently labelled. Fluorescence detection may also include intrinsic fluorescence i.e. detecting tryptophans in proteins.

The detectable analyte may be selected from a fluorescent fusion protein, a fluorescent peptide tag, a fluorescent small molecule or a molecule which forms a fluorescent moiety upon interaction with a protein component. The fluorescent protein may be formed in-situ by complexation of two non-fluorescent species. The fluorescent protein may be for example GFP, YFP, mCherry. The protein may be a fusion of a peptide tag with a protein partner, such as for example peptide tag GFP11 plus protein partner GFP1-10. The detectable analyte may be a fluorescent small molecule such as fluorescein, atto 488 etc. The detectable analyte may be a molecule that forms a fluorescent moiety upon interaction with a protein component, e.g. Orthophthalaldehyde (OPA) reacted with lysine residues (primary amines generally) in the presence of a sulfhydryl. The detectable analyte may be a molecule that forms a fluorescent moiety upon interaction with a nucleic acid component, e.g. an intercalator which fluoresces in the presence of double stranded nucleic acids or a quenched probe that becomes fluorescent in the presence of a particular complementary sequence.

The second droplet contains an aqueous solution. This aqueous solution can be water, a buffer solution or any other aqueous liquid that is not buffered. The aqueous solution does not contain the detectable analyte before mixing, and can be considered as a ‘blank’ solution. A mix of different blank solutions can be used. The blank solution can contain a non-detectable analyte.

The rate of diffusion can be used to calculate the hydrodynamic radius of the biomolecule. This can give an insight in the properties of the biomolecule. As droplets can be merged on demand, the assay can be run repeatedly by merging droplets over a given period of time and measuring the change in the rate of mixing. Protein aggregation over time will cause a decrease in the rate of mixing for example. Thus the stability of the biomolecule can be determined. By defining the temperature at which such a time course is run, the stability of the biomolecule at different target temperatures can be established.

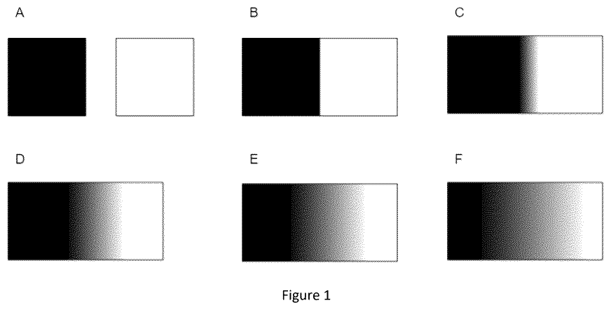

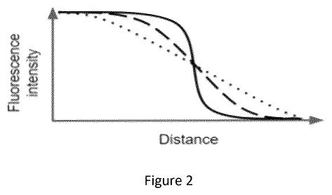

One can measure the advancing front seen in (C) to (F) of FIG. 1 (i.e. the example fluorescence profiles seen in FIG. 2) and calculate the diffusion coefficient of the fluorescent moiety, making necessary assumptions such as the analyte being spherical, originally pure, etc. Then use the following formula to calculate hydrodynamic radius:

$${R}_{H} = {k}_{B} T / 6 π η D$$

Where k_{B} is the Boltzmann constant; T is the temperature; η is the liquid's viscosity; D is the diffusion coefficient.

The liquid's viscosity can be estimated from the literature (e.g. a dilute aqueous protein solution). The temperature can be set and measured.

There may be some non-diffusional based mixing when the two droplets first merge and the surfaces break at the interface. As such, measurement of diffusion may need to have a delayed t=0 point to allow this mixing to cease and wait for pure diffusive mixing to begin.

Assumptions can be made as appropriate for the system and analyte of interest. For example, the analyte can be assumed to be spherical, long rods, short rods, disks, or oblate spheroids.

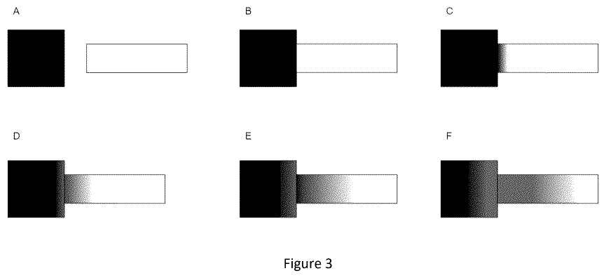

Rather than mixing two square droplets, other geometries may be beneficial. For example, as presented in FIG. 3. Thus one droplet may be square, and one rectangular. The square area may be defined as n×n pixels and the rectangular area n×m pixels. Having a longer blank droplet will give more distance along which diffusion can be measured and may make the image analysis more robust and the calculations more accurate. The gradient shown in FIG. 3 is indicative only and is limited due to the drawing programs used, i.e. diffusion would be continuous, not restarting at the droplet boundary. An additional benefit to controlling droplet geometries is to minimise non-diffusive (e.g. convective) mixing upon merging the droplets. Minimising non-diffusive mixing increases the accuracy and robustness of the approach.

Merging droplets may be performed where the front between the merged droplets is a narrow cross section, for example 2-4 pixels on the device. The connection may be made between two droplets by actuating the space between them. The advancement of the fronts may be controlled by squeezing the sides of the reservoirs. Actuation in the gap and squeezing the sides gives greater control of the speed of the fronts meeting, thereby controlling mixing at the boundary.

The measurement of diffusion can be measured within the stationary droplets. Thus the measurement can be taken for at least 10 minutes in the merged droplet. The hydrodynamic radius can be calculated at least 10 minutes after the droplets are merged. The measurement can be taken for at least 30 minutes in the merged droplet. The hydrodynamic radius can be calculated at least 30 minutes after the droplets are merged.

The non-fluorescent aqueous solution can be any aqueous composition. A selection of different droplets can be used having different properties such as pH, salts or ligands. Thus the properties of the fluorescent biomolecule within a series of different sets of buffers can be measured in the same experiment on the same device.

The use of certain second droplet compositions enables additional parameters and processes to be investigated, for example, diffusiophoresis analysis, i.e. the movement of analytes in in a fluid induced by a concentration gradient of another substance. For example, the other substance may be a salt such as sodium chloride.

Multiple droplets may be merged simultaneously, or in sequence if a time profile is desired. Measurement may be continuous, or may be taken at regular time points during the diffusion process. The multiple droplets may contain the same detectable analyte of interest or a panel of different detectable analytes of interest.

Electrowetting is the modification of the wetting properties of a surface (which is typically hydrophobic) with an applied electric field. Microfluidic devices for manipulating droplets or magnetic beads based on electrowetting have been extensively described. In the case of droplets in channels this can be achieved by causing the droplets, for example in the presence of an immiscible carrier fluid, to travel through a microfluidic channel defined by the walls of a cartridge or microfluidic tubing. Embedded in the walls of the cartridge or tubing are electrodes covered with a dielectric layer each of which are connected to an A/C biasing circuit capable of being switched on and off rapidly at intervals to modify the electrowetting field characteristics of the layer. This gives rise to the ability to steer the droplet along a given path.

As an alternative to microfluidic channel systems, droplets can also be generated and manipulated on planar surfaces using digital microfluidics (DMF). In contrast to channel based microfluidics, DMF utilizes alternating currents on an electrode array for moving fluid on the surface of the array. Liquids can thus be moved on an open-plan device by electrowetting. Digital microfluidics allows precise control over the droplet movements including droplet fusion and separation.

The droplet can be moved using any means of electrokinesis. The droplet can be moved using electrowetting-on-dielectric (EWoD). The electrical signal on the EWoD or optical EWoD device can be delivered through segmented electrodes, active-matrix thin-film transistors, or digital micromirrors. The term electrowetting-on-dielectric (EWoD) device includes a digital microfluidic device having a two-dimensional array of planar microelectrodes, which may be active-matrix electrodes.

The term digital microfluidic device refers to a device having a two-dimensional array of planar microelectrodes. The term excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplet to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminium oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrokinesis occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c \cdot {V}^{2}$$

where θ₀ is the contact angle when the electric field across the interfacial layer is zero, γLG is the liquid-gas tension, c is the specific capacitance (given as εᵣ, ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWoD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/εᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages>100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at a 2-5 microns at the cost of increased voltage requirements for electrowetting. It has also been reported that traditional EWoD devices with parylene C are easily broken and unstable for repeated droplet manipulation with cell culture medium.

Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

### Examples

### Objective

To demonstrate differential rates of diffusion based on molecular size (MW) by bringing droplets containing fluorescent standards of differing molecular weights in contact with a blank buffer. Molecular weight is expected to correlate inversely with the rate of diffusion of each molecule into the adjacent buffer. Diffusion is visualized using time lapse of fluorescent images.
Reagents: —Stock solution of deGFP at a concentration of 2.088 mg/mL
   HNG buffer (20 mM HEPES, 100 mM NaCl, 10% v/v glycerol, pH=7.2)
   Atto 488-Biotin dissolved in buffer (20 mM HEPES-KOH pH 7.2, 50 mM NaCl, 0.1% v/v Tween 20) to a concentration of 0.005 mg/mL
   3% solution of Tween-20
Molecular weight of deGFP: 27.6 kDa
Molecular weight of Atto 488-Biotin: 1.2 kDa

### Fluorescence Measurement Setup:

Exposure settings: Aperture: f/2.8, ISO: 10,000, Shutter speed=¼ s

### Procedure:

Previous Atto-488 data was analyzed to determine concentration of deGFP that would fluoresce at a similar intensity to Atto-488 at a concentration of 0.005 mg/mL. This was to allow both diffusion events to be captured on a single image with an appropriate exposure setting. It was determined that deGFP at 0.5-0.6 mg/mL would exhibit a similar level of fluorescence on-device.

A stock concentration of deGFP was diluted with HNG buffer to appropriate concentration, and this was checked by pipetting 2 μL of both deGFP and Atto-488 onto the top surface of a TFT device and comparing relative fluorescence intensities under the DSLR to ensure that they are similar. deGFP was diluted slightly to match fluorescence of Atto-488.

deGFP, buffer (20 mM HEPES-KOH pH 7.2, 50 mM NaCl, 0.1% v/v Tween 20), and Atto-488 were loaded onto edge 1 of a fresh device to form 3 reservoirs of size 60×50 pixels.

A number of size 10 droplets were dispensed from each reservoir, reshaped to be 5×20 pixel droplets. The fluorescent droplets were brought into contact with a similar droplet of buffer (20 mM HEPES-KOH pH 7.2, 50 mM NaCl, 0.1% v/v Tween 20).

After initial contact, images were captured every 10 seconds for the first five minutes, then every 30 seconds for an additional 10 minutes. Droplets were left actuated for another 5 minutes (for a total of 20 minutes) and another image was taken. Following these 20 minutes of continuous actuation, the script was stopped and the droplets were allowed to “relax” for 3 minutes while images were captured. After this relaxation phase, the droplets were reformed using actuation for another 3 minutes, then again allowed to relax before being actuated a final time. These relaxation/actuation cycles were used to determine whether continuous actuation was slowing diffusion by “holding” molecules in place.

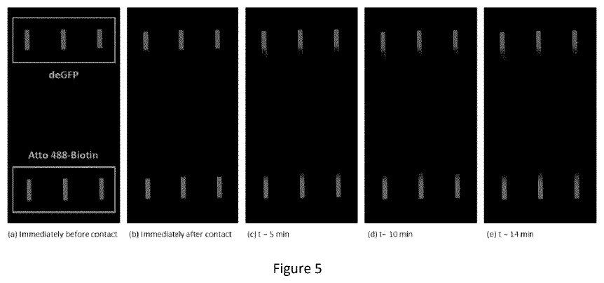

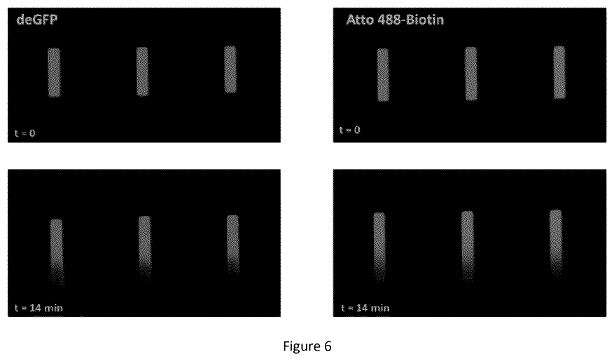

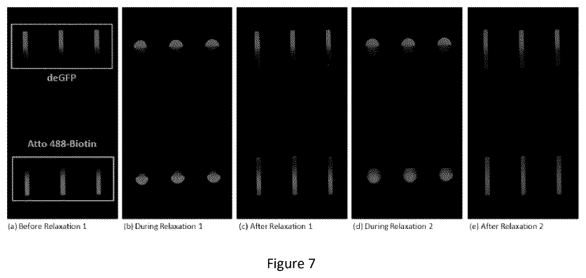

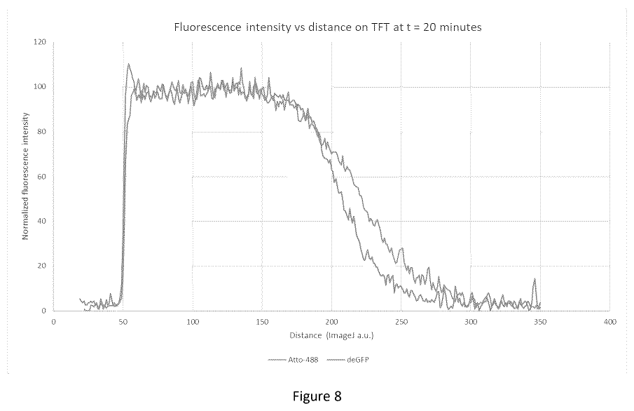

Data is shown in FIGS. 5-8.

The data shows that the difference in behaviour between the deGFP and Atto 488 is clearly apparent. Fluorescent imaging of mixing patterns and rates may therefore be a useful tool in determining the molecular weight of a given reagent.

1. A method for measuring the diffusion within a droplet on an electrowetting on dielectric (EWoD) device, the method comprising:
a. adding at least two droplets onto the device, one droplet containing a detectable analyte and the second droplet containing an aqueous solution;
b. manipulating two droplets to merge the analyte and aqueous solution; and
c. measuring the diffusion of the detectable analyte within the merged droplet and calculating the diffusion coefficient of the detectable analyte.

2. The method according to claim 1, wherein the detectable analyte in the droplet is a single analyte species.

3. The method according to claim 1 or claim 2, wherein the analyte is a biomolecule.

4. The method according to claim 3, where in the biomolecule is a protein, nucleic acid or antibody.

5. The method according to any preceding claim, wherein the analyte is fluorescent.

6. The method according to any preceding claim, wherein the diffusion measurement in step (c) is used to calculate the hydrodynamic radius of the analyte.

7. The method according to claim 5, wherein the hydrodynamic radius is used to calculate the stability of the biomolecule.

8. The method according to claim 5 or claim 6, wherein the hydrodynamic radius is used to detect and/or quantify the interaction of the fluorescent analyte with a non-fluorescent species.

9. The method according to any one of claims 6 to 8, wherein the hydrodynamic radius is calculated at least 10 minutes after the droplets are merged.

10. The method according to any one preceding claim, wherein the device is an AM-EWoD device.

11. The method according to any one preceding claim, wherein the aqueous solution is water, a buffer solution or a non-buffer solution.

12. The method according to any one preceding claim, wherein the detectable analyte is merged with multiple different droplets having differing aqueous solutions.

13. The method according to any one preceding claim, wherein the buffer solution is in the range of pH 7-8 with a total ionic strength of 100-200 mM.

14. The method according to any one preceding claim, wherein the detectable analyte comprises a biomolecule bound to a fluorescent marker.

15. The method according to any one preceding claim, wherein the detectable analyte is selected from a fluorescent fusion protein, a fluorescent peptide tag, a fluorescent small molecule or a molecule which forms a fluorescent moiety upon interaction with a protein component.

16. The method according to any one preceding claim, wherein multiple merged droplets are measured simultaneously.

17. The method according to any one preceding claim, wherein the pre-merged droplets are different shapes.

18. The method according to claim 17, wherein one droplet is square (n×n pixels) and one is rectangular (n×m pixels).

19. The method according to any one preceding claim wherein the presence or absence of a non-detectable analyte in the aqueous droplet is identified by a change in diffusion of the detectable analyte.
